(19) United States
(12) Patent Application Publication
(10) Pub. No.: US 2026/0027135 A1
(43) Pub. Date: Jan. 29, 2026

(54) CANNABINOID FORMULATIONS

(71) Applicant: TAAC NATURALS ENTERPRISES, LLC, Round Rock, TX (US)

(72) Inventors: Allen Cagle, Los Angeles, CA (US); Troy ANDERLOHR, Round Rock, TX (US)

(21) Appl. No.: 18/994,597

(22) PCT Filed: Jul. 13, 2023
(86) PCT No.: PCT/US2023/070145

§ 371 Date: Jan. 14, 2025

Related U.S. Application Data
(60) Provisional application No. 63/368,460, filed on Jul. 14, 2022.

(51) Int. Cl. A61K 31/00; A23L 2/56; A23L 2/60; A23L 2/68; A61K 9/00; A61K 9/08; A61K 9/70; A61K 31/14; A61K 31/198; A61K 31/522; A61K 31/685; A61K 36/185; A61K 36/67; A61K 36/81; A61K 47/10; A61K 47/12; A61K 47/24; A61K 47/36; A61P 39/02

(52) U.S. Cl. CPC A61K 31/658; A23L 2/56; A23L 2/60; A23L 2/68; A61K 9/0019; A61K 9/0053; A61K 9/0095; A61K 9/08; A61K 9/7023; A61K 31/14; A61K 31/198; A61K 31/522; A61K 31/685; A61K 36/3482; A61K 36/67; A61K 36/81; A61K 47/10; A61K 47/12; A61K 47/24; A61K 47/36; A61P 39/02

(57) ABSTRACT

The present invention provides formulations and methods for the treatment of Cannabis toxicity. The formulations can include THCV.

### CLAIM OF PRIORITY UNDER 35 U.S.C. § 119

The present application for patent claims benefit of Provisional Application No. 63/368,460 entitled “CANNIBINOID FORMULATIONS” filed Jul. 14, 2022, and hereby expressly incorporated by reference herein.

### BACKGROUND

### Field

This invention relates to a formulation that can counteract the psychoactive and physiological effects of THC and/or other cannabinoids having effects similar to THC.

### Background

It is not considered possible to have a lethally toxic overdose of Cannabis. However, in part because of its non-lethality in large doses, it is often the case that users consume more than an optimal amount, overshooting the desired effects and, instead, experiencing undesired effects, some of which may be potentially severe. Unwanted effects of Cannabis can include severe anxiety, ataxia/dizziness, sweating, nausea, vomiting, and/or a sensation of dying. Additional symptoms can include disorientation, feeling delirious or feverish, and can be followed by malaise or stupor commonly associated with a “hangover.” In some severe cases, individuals who overdose may also become extremely paranoid, hallucinate, and/or experience panic attacks, creating a risk of potential harm to themselves or others.

### SUMMARY

Embodiments of the invention relate to a formulation for the treatment of Cannabis toxicity comprising nano-emulsified THCV and an emulsifier. In some embodiments, the formulation can include L-theanine.

In some embodiments, the formulation can be in the form of about a 2 oz beverage.

In some embodiments, the formulation can further include a supplement. In some embodiments, the supplement is ashwagandha.

In some embodiments, the formulation can further include an acid. In some embodiments, acid can be one or more of citric acid, lemon juice, tartaric acid, white distilled vinegar, apple cider vinegar, ascorbic acid, and/or the like.

In some embodiments, the formulation includes one or more of 73.536%-99% water, 4.288%-6.432% monk fruit/erythritol, 0.12%-0.18% stevia, 0.104%-0.156% citric acid, 0.56%-0.84% lecithin, 0.056%-0.084 xanthan gum, 0.40%-0.6%, MCT/THCV, 0.072%-0.108% caffeine, 0.144%-0.216% L-theanine, 0.144%-0.216% choline, 0.008%-0.012% black pepper extract, 0.56%-0.84% natural and artificial flavors, 0.008%-0.012% emulsifying system.

In some embodiments, the formulation can be in the form of a transdermal patch. In some embodiments, the formulation can include one or more of THCV, a nanoemulsifying system, alpha-GPC, L-theanine, one or more terpenes, glycol, and/or the like.

In some embodiments, the formulation can be in the form of liquid for IV administration. In some embodiments, the formulation can include one or more of THCV, a nanoemulsifying system, L-theanine, Cannabis-derived lipids, glycerin, water, and/or the like.

In some embodiments, the formulation can be in the form of a liquid for administration with an oral syringe. In some embodiments, the formulation can include one or more of THCV, a nanoemulsifying system, alpha-GPC, L-theanine, one or more terpenes, glycerin, lipids, water, and/or the like.

Some embodiments of the invention relate to a method for manufacturing any of the compositions disclosed herein. In some embodiments, the method can include one or more of: a. cleaning and sanitize a mixing vessel; b. adding the water to the mixing vessel and begin homogenization operation; c. adding one or more of the following ingredients to the mixing vessel: monkfruit/erythritol, stevia, citric acid, lecithin, xanthan gum, MCT/THCV, caffeine, L-theanine, choline, black pepper extract, natural and artificial flavors; d. mixing with a high shear homogenizer until substantially homogeneous; e. adding an emulsifying system as the final ingredient and mixing again with the homogenizer; and/or f. distributing into a container and/or a delivery system (e.g., a syringe or IV bag).

Some embodiments of the invention relate to a method for treating Cannabis toxicity using any formulation provided herein. The method can include administering the formulation to a subject in an amount sufficient to alleviate one or more symptoms of Cannabis toxicity. In some embodiments, the formulation is administered prior to consuming Cannabis. In some embodiments of the method, the formulation is administered after consuming Cannabis.

In some embodiments of the method, the subject can be a human.

In some embodiments of the method, the subject can be an animal.

In some embodiments of the method, the administration step comprises delivering at least 5 mg of THCV.

In some embodiments of the method, wherein the formulation is in the form of an oral composition that can include 50 mg of THCV in 2 fluid ounces.

### DETAILED DESCRIPTION

The present invention relates to a formulation and/or uses of the formulation to help alleviate toxicity symptoms and risks related to negative effects due to the overuse of Cannabis or products derived from Cannabis.

In the methods or processes described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

As used herein, “Cannabis” refers to a genus of flowering plants that includes a single species, Cannabis sativa, which is sometimes divided into two additional species, Cannabis indica and Cannabis ruderalis. These three taxa are indigenous to Central Asia, and South Asia. Cannabis has long been used for fiber (hemp), for seed and seed oils, for medicinal purposes, and as a recreational drug. Various extracts including hashish and hash oil are also produced from the plant. Suitable strains of Cannabis include, e.g., “indica-dominant” (e.g., Blueberry, BC Bud, Holland's Hope, Kush, Northern Lights, Purple, and White Widow), “pure sativa” (e.g., Acapulco Gold and Malawi Gold (Chamba)), and “sativa-dominant” (e.g., Charlotte's Web, Diesel, Haze, Jack Herer, Shaman, Skunk, Sour, and Te Puke Thunder). The Cannabis can include any physical part of the plant material, including, e.g., the leaf, bud, flower, trichome, seed, or combination thereof. Likewise, the Cannabis can include any substance physically derived from Cannabis plant material, such as, e.g., kief and hashish.

As used herein, “Cannabis toxicity” refers to a situation in which a person experiences adverse effects from Cannabis use. As used herein, adverse effects and toxicity symptoms can include paranoia; psychosis; decreased judgment, concentration, perception, and coordination; decreased muscle strength; postural hypotension; lethargy; slurred speech; and/or the like.

As used herein, “pharmaceutical dosage form” refers to a pharmaceutical product in the form in which it is marketed for use, such as the alleviation of Cannabis toxicity, typically involving a mixture of active drug components and nondrug components (excipients), along with other non-reusable material that may not be considered either ingredient or packaging (such as a capsule shell, for example). The term pharmaceutical dosage form can also refer to the chemical formulation of a drug product's constituent drug substance(s) and any blends involved, without considering matters beyond that (such other considerations being, e.g., the ultimate configuration as a consumable product such as a capsule, patch, etc.).

Depending on the method/route of administration, pharmaceutical dosage forms come in several types. These include many kinds of liquid, solid, and semisolid dosage forms. Common pharmaceutical dosage forms include pill, tablet, or capsule, drink, powder to be mixed with water to make a drink, or syrup, and natural or herbal form such as plant or food of sorts, among many others. Notably, the route of administration (ROA) for drug delivery is dependent on the dosage form of the substance in question. A liquid pharmaceutical dosage form is the liquid form of a dose of a chemical compound used as a drug or medication intended for administration or consumption.

Various pharmaceutical dosage forms can exist for a single particular drug, since different medical conditions can warrant different routes of administration. For example, persistent nausea and emesis or vomiting may make it difficult to use an oral dosage form, and in such a case, it may be necessary to utilize an alternate route such as inhalational, buccal, sublingual, nasal, suppository or parenteral. Additionally, a specific pharmaceutical dosage form may be a requirement for certain kinds of drugs, as there may be issues with various factors such as, e.g., chemical stability and/or pharmacokinetics.

Exemplary pharmaceutical dosage forms include, e.g., pills, osmotic delivery systems, elixirs, emulsions, hydrogels, suspensions, syrups, capsules, tablets, orally dissolving tablets (ODTs), gel capsules, thin films, adhesive topical patches, lollipops, lozenges, chewing gum, dry powder inhalers (DPIs), vaporizers, nebulizers, metered dose inhalers (MDIs), ointments, transdermal patches, intradermal implants, subcutaneous implants, transdermal implants, and the like.

As used herein, “oral delivery” or “oral administration” refers to a route of administration wherein the pharmaceutical dosage form is taken through the mouth. Oral administration is a part of enteral administration, which also includes buccal (dissolved inside the cheek), sublabial (dissolved under the lip) and sublingual (dissolved under the tongue) administration. Enteral medications come in various forms, including: tablets to swallow, chew or dissolve in water or under the tongue; capsules and chewable capsules (with a coating that dissolves in the stomach or bowel to release the medication there); time-release or sustained-release tablets and capsules (which release the medication gradually); powders or granules; teas; drops; and liquid medications or syrups.

As used herein, “dermal delivery” or “dermal administration” refers to a route of administration wherein the pharmaceutical dosage form is taken to, or through, the dermis (i.e., layer of skin between the epidermis (with which it makes up the cutis) and subcutaneous tissues).

As used herein, “pill” typically refers to a small, round, solid pharmaceutical oral dosage form of medication that was in use before the advent of tablets and capsules. Pills were historically made by mixing the active ingredients with an excipient such as glucose syrup in a mortar and pestle to form a paste, then rolling the mass into a long cylindrical shape (called a “pipe”), and dividing it into equal portions, which were then rolled into balls, and often coated with sugar to make them more palatable. Today, pills include tablets, capsules, and variants thereof like caplets-essentially anything with medication that can be digested, minus the liquid forms, falls into the pill category.

As used herein, “osmotic delivery system” or “osmotic controlled release oral delivery system” or “OROS” refers to a controlled release oral drug delivery system in the form of a tablet. The tablet typically has a rigid water-permeable jacket with one or more small holes; typically, the small holes are laser-drilled. As the tablet passes through the body, the osmotic pressure of water entering the tablet pushes the active drug through the openings in the tablet.

As used herein, “adhesive topical patch” or “transdermal patch” refers to a medicated adhesive patch that is placed on the skin to deliver a specific dose of medication through the skin and into the bloodstream. A transdermal patch or transdermal system (TDS) is a medicated adhesive patch that is placed on the skin to deliver a specific dose of drug through the skin and into the bloodstream. An advantage of a transdermal drug delivery route over other types of medication delivery such as oral, topical, intravenous, intramuscular, etc. is that the patch provides a controlled release of the medication into the patient, usually through either a porous membrane covering a reservoir of medication or through body heat melting thin layers of medication embedded in the adhesive. The main disadvantage to transdermal delivery systems stems from the fact that the skin is a very effective barrier; as a result, only medications whose molecules are small enough to penetrate the skin can effectively be delivered by this method.

The transdermal patch serves as an alternative dosage form for patients who experience dysphagia (difficulty in swallowing). Additional reasons to use transdermal patches include the convenience of a dosage form that can be taken without water as well as the inability of the patient to cat or drink (e.g., nausea and/or vomiting).

As used herein, “therapeutically effective amount” is intended to include an amount of a substance (or compound) described herein, or an amount of the combination of substances (or compounds) described herein, e.g., to treat or prevent the disease or disorder, or to treat the symptoms of the disease or disorder, in a host, for example by alleviating one or more symptoms of Cannabis toxicity. The combination of substances (or compounds) is preferably a synergistic combination. Synergy, as described for example by Chou and Talalay, Adv. Enzyme Regul., 22:27 (1984) (which is fully incorporated by reference herein), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes: (i) preventing a pathologic condition from occurring; (ii) inhibiting the pathologic condition or arresting its development; (iii) relieving the pathologic condition; and/or (iv) alleviating/diminishing symptoms associated with the pathologic condition. The term alleviate as used herein, can mean to lessen by any degree, for example by 1, 2, 3, 5, 10, 15, 20, 25, 30, 35, 40, 50% or more of a quantified symptom.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

In some embodiments, the formulation can include nano-emulsified THCV. In some embodiments, the formulation provides at least 1 mg of THCV per dose. For example, the formulation can provide 1, 2, 3, 4, 5, 10, 15, 20, 25, 30, 35, 40, 45, 50, 55, 100, 200, 300, 400, 500, 600, 700, 800, 900, 1,000 or more mg per dose. A “dose” refers to the amount used in a single administration of the formulation to a subject. In some embodiments, the formulation includes 0.0001%-99% THCV or 1%-50% THCV. For example, the formulation can include about 0.0001, 0.001, 0.002, 0.003, 0.1, 0.5, 1, 5, 10, 15, 20, 25, 50% or more THCV.

As used herein, “nano-emulsified” can refer to using an emulsifying agent (also referred to as an emulsifier) to mix two immiscible liquids and form a single-phase liquid. The resulting single-phase liquid can contain an emulsion structure(s) less than 100 nm in diameter. In some embodiments, the liquid can contain an emulsion structure of less than 100, 80, 60, 40, or 20 nm, or less. In other embodiments, the emulsion structure is 120, 150, or 200 nm, or more.

The formulation can include but is not limited to THCV, an emulsifier, and/or a base.

Non-limiting examples of emulsifiers can include, but are not limited to, lecithin (e.g., sunflower lecithin), guar gum, egg yolk, mustard extract, and/or the like, and/or combinations thereof. In some embodiments, the formulation includes 0.001%-60% emulsifier(s). For example, the formulation can include 0.001, 0.01, 0.1, 0.5, 1, 10, 25, 30, 35, 40, 45, 50, 55, 60% or more emulsifier(s).

Non-limiting examples of the base can be water, any MCT oil, and/or the like. The MCT oil can be, but is not limited to hempseed oil, grapeseed oil, coconut oil, palm kernel oil, sunflower oil, olive oil, sesame oil, avocado oil, palm oil, soybean oil, corn oil, peanut oil, canola oil, corn oil, hazelnut oil, rice bran oil, linseed oil, safflower oil, sesame oil, passionfruit oil, fish oil, and/or the like. In some embodiments, the formulation includes 0.01%-99.5% base. For example, the formulation can include 0.01, 0.1, 0.5, 1, 10, 25, 30, 35, 40, 45, 50, 55, 60, 65, 70, 75, 75, 80, 85, 90% base.

In some embodiments, the formulation can include a nanoemulsion of an oil, such as MCT, and THCV. Oils that can be used include, but are not limited to, vitamin E; vitamin B12; vitamin A; vitamin D; vitamin B; omega 3; astaxanthin; fish oil; long chain triglyceride (LCT) oil; coconut oil; palm oil; eicosapentaenoic acid (EPA); docosahexaenoic acid (DHA); essential oils such as but not limited to lemon oil, orange oil, peppermint oil, Ylang-Ylang oil, lemongrass oil, tea tree oil, rosemary oil, Australian sandalwood oil, grapefruit oil, frankincense oil, cedarwood oil, patchouli oil, cinnamon bark oil, bergamot oil, chamomile oil, lemon-eucalyptus oil, ginger oil, key lime oil, vanilla oil, clove oil; any other essential oil; and any combination thereof. I

In some embodiments, the formulation can include one or more terpenes. The terpene can be myrcene, limonene, linalool, beta-caryophyllene, alpha-pinene and beta-pinene, alpha-bisabolol, eucalyptol, trans-nerolidol, humulene, delta-3-carene, camphene, borneol, terpineol, valencene, geraniol, eugenol, sabinene, phellandrene, borneol, isoborneol, phytol, menthol, geraniol, citronellol, ocimene, halomon, thymol, carvacrol, thujene, camphene, camphor, verbenone, botrydial, ngaione, cuparane, labdane, ferruginol, cafestol, any other terpene, and any combination thereof. In some embodiments, the formulation can provide 1, 2, 3, 4, 5, 10, 15, 20, 25, 30, 35, 40, 45, 50, 55, 100, 200, 300, 400, 500, 600, 700, 800, 900, 1,000 or more mg of terpenes per dose. In some embodiments, the formulation includes 0.0001%-50% terpenes. For example, the formulation can include about 0.0001, 0.001, 0.01, 0.1, 0.5, 1, 5, 10, 15, 20, 25, 50% or more terpenes.

In some embodiments, the formulation can include one or more lipids or Cannabis-derived lipids or hemp derived lipids. For example, such lipids include waxes and other lipids naturally included in Cannabis. In some embodiments, the formulation includes 0.0001%-80% lipids. For example, the formulation can include about 0.0001, 0.001, 0.002, 0.003, 0.01, 0.1, 0.5, 1, 5, 10, 15, 20, 25, 30, 40, 50% or more lipids.

In some embodiments, two or more components of the formulation can have a synergistic effect. The synergistic effect can be the alleviation of a symptom of Cannabis toxicity. For example, the formulation can include THCV and a terpene, which can have a synergistic effect to alleviate one or more symptoms of Cannabis toxicity.

The THCV can be nanoemulsified using an emulsification system. As defined herein, an “emulsification system” or “emulsifying system” or “emulsifying matrix” or “nano emulsifier” or “nano emulsifier complex” can refer to a system that comprises a matrix that is homogenous in both oil and water-based products wherein an active ingredient can be loaded into the matrix and nano-emulsifed to increase absorption and product stability and bioavailability. An example of such a system is SENDS™ (Self-Emulsifying Nano-Delivery System). Through this disclosure, wherever SENDS™ is described, an alternative emulsifying system can be used instead (or in addition to).

In some embodiments, the formulation can include a base to bring the formulation to a desired volume. The base can be water, any MCT oil, and/or the like. For example, the base can be olive oil, sunflower oil, coconut oil, canola oil, corn oil, soybean oil, peanut oil, sesame oil, safflower oil, grapeseed oil, almond oil, walnut oil, hazelnut oil, pistachio oil, macadamia nut oil, avocado oil, flaxseed oil, pumpkin seed oil, hemp seed oil, rice bran oil, camelina oil, mustard oil, argan oil, black seed oil, pecan oil, cashew oil, marula oil, apricot kernel oil, squalane oil, emu oil, MCT (medium chain triglycerides) oil, fish oil (often consumed in supplement form), krill oil (also consumed in supplement form), evening primrose oil, borage oil, wheat germ oil, black currant seed oil, chia seed oil, rosehip oil, neem oil (used more commonly in topical applications), glycerin, glycol propylene glycol, sorbitol, polyethylene glycol (PEG), butylene glycol, ethylene glycol, xylitol, mannitol, isopropyl myristate, caprylic/capric triglyceride, propanediol, sodium lactate, sodium pca (pyrrolidone carboxylic acid), dimethicone, cyclomethicone, cetyl alcohol, stearyl alcohol, behenyl alcohol, glycereth-26, glycereth-7, glycereth-20, butyrospermum parkii (shea butter), lanolin, jojoba oil, ceramides, hyaluronic acid, soy lecithin powder, sunflower lecithin powder, tocopherol (vitamin e) powder, xanthan gum powder, guar gum powder, acacia gum (gum arabic) powder, sodium caseinate powder, microcrystalline cellulose powder, maltodextrin powder, sodium stearoyl lactylate (SSL) powder, sodium alginate powder, polyglycerol esters of fatty acids (PGE) powder, polysorbate 80 powder, carboxymethyl cellulose (cmc) powder, gelatin powder, modified starches (e.g., cornstarch, potato starch) powder, sodium carboxymethylcellulose (Na-CMC) powder, magnesium aluminum silicate powder, calcium caseinate powder, whey protein powder polysorbate 80, cetearyl alcohol, glyceryl stearate, sorbitan oleate, stearic acid, cetyl alcohol, sodium stearoyl lactylate, PEG-40 hydrogenated castor oil, PEG-100 stearate, lecithin, glycerin, beeswax, xanthan gum, carbomer, polyglyceryl-3 methylglucose distea, and/or the like. the MCT oil can be, but is not limited to hempseed oil, grapeseed oil, coconut oil, palm kernel oil, sunflower oil, olive oil, sesame oil, avocado oil, palm oil, soybean oil, corn oil, peanut oil, canola oil, corn oil, hazelnut oil, rice bran oil, linseed oil, safflower oil, sesame oil, passionfruit oil, fish oil, and/or the like. In some embodiments, the formulation includes 0.01%-99.5% base. For example, the formulation can include 0.01, 0.1, 0.5, 1, 10, 25, 30, 35, 40, 45, 50, 55, 60, 65, 70, 75, 75, 80, 85, 90% base.

In some embodiments, the formulation can include a sweetener. The sweetener can be caloric or non-caloric. Non-limiting examples of a sweeteners can include, but are not limited to, monk fruit, erythritol, sugar, agave nectar, honey, sorghum, stevia, sucralose, acesulfame k, neotame, advantame, aspartame, saccharin, sucralose, and/or the like, and/or combinations thereof. In some embodiments, the formulation can include 0.0001%-50% sweetener. For example, the formulation can include 0.0001, 0.001, 0.01, 0.1, 0.5, 1, 10, 25, 30, 35, 40, 45, 50% or more sweetener.

In some embodiments, the formulation can include an acid. The acid can act as a preservative and/or can be added for taste. Non limiting examples of acids can include, but are not limited to, citric acid, lemon juice, tartaric acid, white distilled vinegar, apple cider vinegar, ascorbic acid, and/or the like, and/or combinations thereof. 0.0001%-50% thickener. For example, the formulation can include 0.0001, 0.001, 0.01, 0.1, 0.5, 1, 10, 25, 30, 35, 40, 45, 50% or more thickener.

In some embodiments, the formulation can include a thickener. Non-limiting examples of a thickener can include, but are not limited to, xanthan gum, psyllium husk, chia seed, flax seed, cornstarch, gelatin, egg whites, agar agar, and/or the like, and/or combinations thereof. 0.0001%-50% thickener. For example, the formulation can include 0.0001, 0.001, 0.01, 0.1, 0.5, 1, 10, 25, 30, 35, 40, 45, 50% or more thickener.

In some embodiments, the formulation can include an additional ingredient or a supplement such as but not limited to ashwagandha, caffeine, chicory root, one or more B vitamins, carob, Rhodiola, maca, peppermint, Cordyceps, ginseng, berry extract, yerba matte extract, L-theanine, epigallocatechin gallate (EGCG), choline, alpha GPC, and/or the like, and/or combinations thereof. For example, the formulation can provide 1, 2, 3, 4, 5, 10, 15, 20, 25, 30, 35, 40, 45, 50, 55, 100, 200, 300, 400, 500, 600, 700, 800, 900, 1,000 or more mg of a supplement per dose. In some embodiments, the formulation can include 0.0001%-50% supplements. For example, the formulation can include about 0.0001, 0.001, 0.01, 0.1, 0.5, 1, 5, 10, 15, 20, 25, 50% or more supplement(s).

In some embodiments, the formulation can include natural and/or artificial flavors. In some embodiments, the formulation can include 0.0001%-50% artificial flavor. For example, the formulation can include about 0.0001, 0.001, 0.01, 0.1, 0.5, 1, 5, 10, 15, 20, 25, 50% or more flavor(s).

In some embodiments, the formulation can contain a dosage of about 0.001 g 15 g THCV (e.g., 0.001 or less, 0.001, 0.01, 0.1, 1, 5, 7, 9, 11, 13, 15 g, or more THCV). In other embodiments, the formulation can contain a dosage of about 0.0001 mg-20 g THCV (e.g., 0.0001 or less, 0.001, 0.01, 0.1, 1, 3, 5, 10, 15, 20 or more g).

### Small Volume Beverage Formulation

In some embodiments, the formulation is formulated into a small volume beverage for consumption. For example, the beverage can be 1, 2, 3, 4, or 5 oz. The beverage formulation can include a sweetener, and natural and/or artificial flavors. The beverage formulation can include a thickener to obtain a desired consistency. Optionally, the formulation can include a supplement. The supplement can be any supplement known in the art to increase energy, improve muscle recovery, improve immune support, improve heart health, supply antioxidants, contain anti-inflammatory properties, improve concentration, improve cognition, and/or the like.

In some embodiments, 1 mg-15 g THCV (e.g., 0.001, 0.001, 0.01, 0.1, 1, 5, 7, 9, 11, 13, or 15 g THCV) can be delivered per beverage. Larger volume beverages are also part of the invention. The beverage can be 5, 6, 7, 8, 9, 10, 11, 12, 13, 14, 15, 16 oz or more.

In some embodiments, the beverage can include one or more of THCV, an emulsifier, a sweetener, a supplement (e.g., caffeine), and/or one or more flavoring agents. In some embodiments, the beverage can include black pepper extract. In some embodiments, the beverage can include nano-emulsified THCV, MCT, sweetener, citric acid, lecithin, xanthan gum, L-theanine, choline or alpha GPC, black pepper extract, natural and artificial flavors, and/or the like.

### Transdermal Patch Formulation

In some embodiments, the formulation is in the form of a transdermal patch.

The topical adhesive patch can include a backing having a front side and a back side. The patch includes a formulation that is in contact with the front side of the backing. The formulation can include an adhesive and Cannabis concentrate.

Since the backing can be porous and/or vapor permeable, many consumers typically refer to the device as a “patch,” a “skin patch,” a “topical patch,” “a transdermal patch” or an “adhesive skin patch.” As such, the device will herein be referred interchangeably to as a patch, a skin patch, and/or an adhesive skin patch. It is appreciated that those skilled in the art understand that the term “patch” is used to refer to the device and is not otherwise limiting in any manner.

The topical adhesive patch can be manufactured in a manner, employing suitable ingredients, such that any one or more of the desired pharmacokinetic metrics (e.g., dose, area under the curve, peak plasma concentration, dosing intervals, time to reach peak plasma concentration, clearance, bioavailability, etc.) are achieved. For example, the topical adhesive patch can be manufactured such that the topical adhesive patch provides for an immediate release (IR) or a time-release (e.g., controlled release (CR), modified release (MR), extended release (ER), or combination thereof) of active ingredient. The topical adhesive patch described herein therefore possesses the potential to allow the development of sensitive drug targets that may otherwise not be feasible in tablet or liquid formulations.

### Backing

The backing is defined by a front side (the side exposed to the subject, or an article of clothing of the subject, during use) and a back side (the side exposed to the environment during use). The backing should be nonirritating to human skin. The backing is a self-supporting sheet of water soluble or water insoluble, polymeric or natural material that provides strength and integrity for the formulation. The backing of the adhesive patch can be vapor permeable. The backing can also be porous, since porosity provides openings for receiving the formulation, and it helps to assure that the adhesive skin patch is vapor permeable. Specifically, the backing can retain the formulation while allowing moisture from the skin to pass. Alternatively, the backing can be non-porous. The backing can have any suitable thickness. In specific embodiments, the suitable thickness allows for a flexible, bendable, pliable, vapor permeable, and/or a stretchable sheet of water insoluble porous material. Specifically, the thickness of the backing can be about 0.001 mm to about 5.0 mm, about 0.001 mm to about 3.0 mm, or about 0.025 mm to about 1.25 mm.

The backing can be manufactured from any suitable material. In specific embodiments, the suitable material forms a flexible, bendable, pliable, and/or stretchable backing. The backing includes a porous or non-porous sheet of water soluble or water insoluble material that provides support for the adhesive skin patch. The backing can include water soluble or water insoluble polymeric fibers, a porous film, or any other kind of matrix with spaces within the matrix. A specific backing is a lightweight, porous, pliable strip composed of a nonwoven fabric of polymeric or natural fibers such as polyester, cotton, or cellulose fibers bonded together with a sizing resin. The backing can be woven or nonwoven. In one embodiment, the backing includes nonwoven fabric. Specifically, the backing can include polyester fibers, polyurethane fibers, polyolefin fibers, polyamide fibers, natural fibers, cotton fibers, copolyester, copolyester fibers, cellulose acetate fibers, polycellulose fibers, or any mixture thereof. Additional stable, water insoluble flexible sheet materials and methods for manufacturing suitable backings are disclosed, e.g., in U.S. Pat. Nos. 4,675,009; 5,536,263; 4,696,854; 5,741,510; and references cited therein, are suitable as backings according to the present invention. The infusion of the formulation into the backing can be accomplished, e.g., with the use of a continuous process mixer, as disclosed, e.g., in U.S. Pat. No. 5,536,263, and references cited therein; or as discussed herein. All of the foregoing references are fully incorporated by reference herein.

In a specific embodiment, the backing can be manufactured from a substance or substances that are generally recognized as safe (GRAS) for topical use.

The backing can be manufactured from a suitable non-woven fabric that is commercially available.

Alternatively, the fibers of the backing can be interlocked mechanically by air or water.

The backing can include a front side and a back side. The adhesive skin patch can include a formulation located in at least a portion of the front side of the backing, on at least a portion of the front side of the backing, or on and in at least a portion of the front side of the backing. As such, the formulation can be located on the entire surface of the front side of the backing, or the formulation can be located on a portion of the surface of the front side of the backing.

In one embodiment, the formulation can be located on the entire surface of the front side of the backing. In addition to being located on the surface of the front side of the backing, the formulation can be located in at least a portion of the underlying surface of the front side of the backing (e.g., the formulation can be partially embedded into the backing).

The formulation can penetrate a substantial portion of the front side of the backing, as disclosed, e.g., in U.S. Pat. No. 5,536,263, and references cited therein, which are all fully incorporated by reference herein. For example, the formulation can penetrate about one-tenth to about nine-tenths the thickness of the backing, or about one-fourth to about nine-tenths the thickness of the backing. As such, the formulation can be partially embedded into the backing. In one embodiment, the formulation can be located on the entire front side of the backing and partially in the front side of the backing (e.g., the formulation is partially embedded into the backing).

Alternatively, a portion of the front side of the backing can include the formulation and other portions of the front side of the backing can include any suitable and effective combination of the pressure sensitive adhesive and, optionally, the solvent. For example, a central circular portion of the front side of the backing can include the formulation while the remaining portions of the front side of the backing include only the pressure sensitive adhesive. The formulation, when partially embedded into the front side of the backing, can impart strength and structure into the adhesive patch. For example, when the formulation is partially embedded into the backing, the likelihood that the adhesive patch tears apart when separated from the release liner or when removed from the skin after use, is lowered.

When the adhesive skin patch is placed upon the skin of a subject (e.g., a human, a canine, or the like), the formulation can be in continuous contact with the skin surface of the subject.

In one embodiment, the adhesive skin patch, upon contact with skin, can allow the skin to breathe. In one embodiment, the adhesive skin patch, upon prolonged contact with skin, holds in place the formulation, and permits the skin to breathe over prolonged periods of time typically experienced with the use of the patch, e.g., up to about 7 days, up to about 24 hours, up to about 12 hours, up to about 8 hours, or up to about 6 hours.

The adhesive skin patch can be reversibly attached to a release liner. The release liner helps to maintain the adhesiveness of the adhesive skin patch prior to use, such as during manufacturing, packaging, shipping, and/or storage. Any suitable release liner can be employed for use in the present invention. Suitable release liners 10 are readily known to those of skill in the art. Sec, e.g., U.S. Pat. Nos. 4,675,009; 5,536,263; 4,696,854; 5,741,510; and references cited therein for further descriptions of release liners 10 useful in the present invention. All of the foregoing references are fully incorporated by reference herein. The release liner can include a perforation that allows the tab section of the release liner to be removed. Removal of the tab section of the release liner can allow the adhesive skin patch to be removed from the release liner with relative ease.

The backing can be a porous or non-porous, self-supporting sheet of water insoluble or water soluble, polymeric or natural material that provides strength and integrity for the formulation. For example, the backing can be water insoluble polymeric fibers, open cell foam backing (e.g., polyurethane, polyvinyl chloride, or polyethylene), a porous film, or any other kind of matrix with spaces within the matrix. In one embodiment, the backing can include polyester, polyurethane, polyolefin, polyamide fibers, natural fibers, cotton fibers, polycellulose fibers, or any mixture thereof.

The back side of the backing of the patch can be relatively dry to the touch, such that upon contact, e.g., with a skin surface or article of clothing, no appreciable or significant amount of liquid, gel, ointment, fluid, lotion, and the like, present in the back side of the backing of the patch is transferred there from and deposited upon the skin surface or article of clothing.

The back side of the backing of the patch can have a relatively small degree of moisture, while still being considered a “dry” patch, and would still be relatively dry to the touch, such that upon contact, e.g., with a skin surface or article of clothing, no appreciable or significant amount of liquid, gel, ointment, fluid, lotion, and the like, present in the back side of the backing of the patch is transferred there from and deposited upon the skin surface or article of clothing.

The size of the patch can be about 1-10 square inches (6.5 to 65 square centimeters). For example, the patch can be 1, 1.5, 1.75, 2, 4, 6, 8, 10 square inches.

In some embodiments, 1 mg-15 g THCV (e.g., 0.001, 0.001, 0.01, 0.1, 1, 5, 7, 9, 11, 13, or 15 g THCV) can be delivered per patch. A single patch can include a single dose/dosage.

In some embodiments, the formulation used to manufacture the patch can include one or more of THCV, an emulsifier, alpha-GPC, L-theanine, terpenes, glycol, and/or the like.

### IV Therapy

In some embodiments, the formulation is formulated for use by IV administration. For example, the formulation is 100, 150, 200, 300, 400, 500, 600 mL or more liquid suitable for IV administration in a bag.

A formulation suitable for parenteral administration can include a sterile aqueous preparation of the active agent, or a dispersion of a sterile powder of the active agent, which is preferably isotonic with the blood of the subject. Parenteral administration of a pharmaceutical composition (for example, through an IV drip) is one form of administration. An isotonic agent may be included in the liquid preparation including, for example, a sugar; a buffer; and/or a salt including, for example, sodium chloride. A solution of the active agent may be prepared in water, optionally mixed with a nontoxic surfactant. A dispersion of the active agent may be prepared in water, ethanol, a polyol (such as glycerol, propylene glycol, liquid polyethylene glycols, and the like), a vegetable oil, or a glycerol ester, or a mixture thereof. The ultimate dosage form may be sterile, fluid, and stable under the conditions of manufacture and storage. The necessary fluidity may be achieved, for example, by using liposomes, by employing the appropriate particle size in the case of dispersions, or by using surfactants. Sterilization of a liquid preparation may be achieved by any convenient method that preserves the bioactivity of the active agent, preferably by filter sterilization. Methods for preparing a powder include vacuum drying and freeze drying of the sterile injectable solutions. Subsequent microbial contamination may be prevented using various antimicrobial agents, for example, antibacterial, antiviral and antifungal agents including parabens, chlorobutanol, phenol, sorbic acid, thimerosal, and the like. Absorption of the active agents over a prolonged period may be achieved by including agents for delaying, for example, aluminum monostearate and gelatin.

As used herein “lipid emulsion therapy” is a medical treatment that involves the infusion of a therapeutic formulation into a subject's bloodstream through an IV line.

Lipid emulsion therapy can include a mixture of different types of fats (lipids) that are suspended in a solution that can be administered intravenously (IV). The exact composition of the lipid emulsion can vary depending on the specific product used and the condition being treated, but it generally contains a combination of the following components:

In some embodiments, soybean oil: This is a common source of fat used in lipid emulsions, as it is high in polyunsaturated fatty acids, which are thought to have beneficial effects in the body.

In some embodiments, egg phospholipids: These are naturally occurring compounds found in egg yolks that help to stabilize the lipid emulsion and make it easier to administer.

In some embodiments, glycerol: This is a sugar alcohol that is added to the emulsion to help maintain its stability and prevent it from separating into its component parts.

Purified water: This is used as a base for the emulsion and helps to ensure that it can be easily infused into the patient's bloodstream.

In some embodiments, 1 mg-15 g THCV (e.g., 0.001, 0.001, 0.01, 0.1, 1, 5, 7, 9, 11, 13, or 15 g THCV) can be delivered per bag.

In some embodiments, the formulation for IV administration can include one or more of THCV, an emulsifier, L-theanine, terpenes, Cannabis-derived lipids, glycerin, water, and/or the like.

### Oral Syringe

In some embodiments, the formulation is formulated for oral administration. For example, the formulation is 5, 10, 15, 20, 25 mL or more liquid suitable for oral administration in a syringe.

In some embodiments, 1 mg-15 g THCV (e.g., 0.001, 0.001, 0.01, 0.1, 1, 5, 7, 9, 11, 13, or 15 g THCV) can be delivered per syringe. In some embodiments, a single syringe is a single dose/dosage.

In some embodiments, the oral syringe can include one or more of THCV, an emulsifier, alpha-GPC, L-theanine, terpenes, glycerin, lipids, water, and/or the like.

### Pharmaceutical Formulation

In some embodiments, the formulation is formulated into any pharmaceutical dosage for administration in a subject. The subject can be human or an animal. For example, the formulation can be administered to a small animal, such as a dog that has inadvertently consumed a substance containing Cannabis or extracts thereof. For example, the formulation can be administered orally in a syringe.

### Other Forms

It is a further object of the present invention to disclose the formulation as defined in any of the above, wherein said formulation is formulated in a dosage form selected from the group consisting of liquid, solid, gas, oral, sublingual, pill, tablet, capsule, buccal, tincture, strip, film, spray, lozenge, effervescent form, sublingual, granules, orally-disintegrating, thin film, liquid, solution, suspension, emulsion, powder or liquid or solid crystals, powder, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, syrup, skin patch, vaginal, suppository, pessary, rectal and any combination thereof.

### Methods of Using

Some embodiments of the invention relate to methods for using the formulation.

In some embodiments, the method alleviates toxicity symptoms related to the overconsumption of a product containing Cannabis or an extract thereof. In some embodiments, the method can include administration of the formulation to a subject in a dosage sufficient to cause alleviation of one or more symptoms.

In some embodiments, the method enhances cognition.

Administration can be oral administration, transdermal administration, intravenous administration, or the like.

The alleviation of one or more symptoms can occur within 1, 2, 3, 4, 5, 10, 15, 20, 30 45, 60 min or more of administration. The method can include delivering a single dose or multiple doses over a period of time. The period of time can be 1, 6, 12, 18, 24, or more hours.

### Methods of Manufacturing

Some embodiments of the invention relate to methods of manufacturing any of the formulations described herein. In some embodiments, the method includes adding all the ingredients together and homogenizing by means of a homogenizer, mixer, or any such device commonly used in the art. The method can include adding a self-emulsification system such as SENDS™.

In some embodiments, the method can include one or more steps of: 1. sanitizing all lab equipment; 2. adding a base (e.g., glycol or glycerin) to a container such as a beaker; 3. heating and mixing the base until it reaches of temperature of about 150° C. (In some embodiments, the temperature can be 100° C., 110° C., 120° C., 130° C., 140° C. or more. This step can ensure proper fluidity and facilitate the dissolution process); adding THCV (tetrahydrocannabivarin) to the beaker containing the base and mixing until THCV is dissolved; adding a nano-emulsifying system to the mixture in the beaker and mixing for 30 minutes (in some embodiments, the mixture is mixed for at least 5, 10, 15, 20, 25, or more minutes; any suitable mixture can be used, for example a high shear mixer). In some embodiments the method includes adding alpha GPC and L-theanine to the mixture and mixing for an additional 10 minutes (in some embodiments, the mixture is mixed for at least 2, 5, 7 or more min). In some embodiments, the method includes adding terpenes and/or any additional ingredients. The terpenes and additional ingredients are mixed into the mixture. Once all the components have been substantially mixed and integrated, the heat is turned off turn off. In some embodiments, the mixing steps occur at a temperature of 100° F., 120° F., 150° F., 200° F., 250° F., 300° F., 350° F., 380° F. or more. In some embodiments at this point, the formulation is complete and ready for use in a desired form (e.g., transdermal patch reservoir, syringe, etc.).

### EXAMPLES

### Example 1

This example provides an exemplary formulation of the invention, wherein the formulation is in the form of about a 2 oz beverage that contains 50 mg THCV.

TABLE 1

| Ingredient | % |
|---|---|
| Water | q.s. or 91.92 |
| Monkfruit/Erythritol | 5.36 |
| Stevia | 0.15 |
| Citric Acid | 0.13 |
| Lecithin | 0.70 |
| Xanthan Gum | 0.07 |
| 4 MCT: 1 THCV | 0.50 |
| Caffeine | 0.09 |
| L-Theanine | 0.18 |
| Choline or alpha GPC | 0.18 |
| Black Pepper Extract | 0.01 |
| Natural and artificial flavors | 0.70 |
| SENDS ™ | 0.01 |

### Example 2

This example provides a method of making the formulation of Example 1:
1. Clean and sanitize a mixing vessel. The mixing vessel can be any container suitable to hold all the ingredients.
2. Add the water to the mixing vessel and begin homogenization operation.
3. Stepwise add the following ingredients to the mixing vessel: monkfruit/erythritol, stevia, citric acid, lecithin, xanthan gum, MCT/THCV, caffeine, L-theanine, choline, black pepper extract, natural and artificial flavors
4. Mix with a high shear homogenizer until thoroughly homogeneous. This will vary depending on batch size.
5. Add emulsifier system as the final ingredient and mix again with the homogenizer.
6. Distribute into aluminum containers and begin cleaning processes.

### Example 3

Experiments are done to compare a formulation containing nano-emulsified TCHV and a formulation containing non-nano-emulsified THCV. It was found that the formulation with the nano-emulsified THCV is more effective.

### Example 4

A formulation in the form for subcutaneous injection in a small animal was used and found to alleviate one or more symptoms of Cannabis toxicity in dogs within 20-30 minutes of administration.

The following is an observation made by a veterinarian: “On Jun. 13, 2022, “Whiskey”, a 10-year-old, M-N, Australian Shepherd presented for Cannabis ingestion/toxicity (Approx. 300 mg THC per Owner). Owner stated that they were treating another dog in the household with a topical THC oil treatment and this patient consumed it orally from licking off housemate. He was lethargic and had bilaterally injected sclera. 25 mg of THCV in HiBegone (half dose) was administered orally, and the patient returned to normal cognitive function in 15 minutes and was completely normal in 30 minutes. After a third recurrence including same symptoms and dosages, Owner started administering a preventative dose of 25 mg THCV of HiBegone 15 minutes prior to treatment of housemate and that proved to prevent all potential symptoms.” “HiBegone” or “HIBEGONE” is the formula described in Example 1.

### Example 5

This example describes the components for one embodiment of a 2 oz liquid beverage:
a. Water—73.536%-99.99%
b. Monkfruit/Erythritol—4.288%-6.432%
c. Stevia—0.12%-0.18%
d. Citric Acid—0.104%-0.156%
e. Lecithin—0.56%-0.84%
f. Xanthan Gum—0.056%-0.084
g. MCT/THCV (ratio of 4:1)—0.40%-0.6%
h. Caffeine—0.072%-0.108%
i. L-Theanine—0.144%-0.216%
j. Choline—0.144%-0.216%
k. Black Pepper Extract—0.008%-0.012%
l. Natural and artificial flavors—0.56%-0.84%
m. SENDS™—0.008%-0.012%

### Example 6

The following is a table of specific concentrations of ranges of three different embodiments of the invention. These are only examples, and the invention includes variations on these concentrations. For example, the embodiments can be plus or minus 0.01, 0.05, 0.1, 0.25, 0.5, 0.75, 1, 2, 5, 10, 15, 20, 25%, 30%, 40%, 50%, 60% or more or less of the concentrations in the table. The embodiments can be plus 100%, 125%, 150%, 200% or more of the concentrations in the table. The ingredients listed in the table are optional and can be removed or replaced with a similar ingredient. The ratios of the ingredients can vary.

TABLE 2

| | | |
|---|---|---|
| Ingredient | % | mg |

Transdermal Patch

| | | |
|---|---|---|
| THCV | 0.0026 | 40 |
| Nano-emulsifier | 0.03 | 450 |
| Alpha-GPC | 0.0066 | 100 |
| L-theanine | 0.0066 | 100 |
| Terpenes | 0.00066 | 10 |
| Glycol | 50 | — |
| Water | q.s. | — |

25% HBG Lipid Solution (500 ml bag) 125 ml active

| | | |
|---|---|---|
| THCV | 0.00032 | 40 |
| Nano-emulsifier complex | 0.0036 | 450 |
| L-theanine | 0.0008 | 100 |
| Canna/Hemp derived lipids | 2.5 | — |
| Glycerin | 2.5 | — |
| Water | q.s. | — |

15 ml Oral Syringe

| | | |
|---|---|---|
| THCV | 0.0026 | 40 |
| Nano-emulsifier | 0.03 | 450 |
| Alpha-GPC | 0.0066 | 100 |
| L-theanine | 0.0066 | 100 |
| Terpenes | 0.00066 | 10 |
| Glycerin | 20 | — |
| Lipids | 20 | — |
| Water | q.s. | — |

### Example 7

### Transdermal Patch

The following is an example method for making an embodiment of the invention in the form of a transdermal patch.

Sanitize all lab equipment: Before starting the formulation process, ensure that all equipment, including beakers, mixers, and any other utensils, are thoroughly sanitized.

Prepare the mixture. Begin by adding glycerin to a beaker. Heat the beaker and mix the glycol until it reaches a temperature of 150° C. This step ensures proper fluidity and facilitates the dissolution process.

Dissolve THCV: Once the glycol has reached the desired temperature, add THCV (tetrahydrocannabivarin) to the beaker. Allow the mixture to continue mixing until the THCV is fully dissolved. This step ensures proper dispersion of THCV in the solution.

Incorporate nano complex: Add the nano complex to the mixture in the beaker. Use a high shear mixer and continue mixing for 30 minutes. This ensures uniform distribution of the nano complex throughout the formulation, enhancing its effectiveness.

Integrate alpha GPC and L-theanine: After the nano complex is well incorporated, add alpha GPC and L-theanine to the mixture. Allow the solution to mix for an additional 10 minutes.

Mix in terpenes: Next, add the desired terpenes to the mixture. Mix the formulation to ensure even distribution of the terpenes throughout the solution.

Turn off heat: Once all the components have been properly mixed and integrated, turn off the heat source.

Packaging: At this point, the formulation is complete and ready injected into the reservoir of the transdermal patch.

### Example 8

### Lipid Solution IV Formulation

The following is an example method for making a formulation suitable for IV administration.

Sanitize all lab equipment: Before starting the formulation process, ensure that all equipment, including beakers, mixers, and any other utensils, are thoroughly sanitized.

Prepare the mixture: Begin by adding glycerin to a beaker. Heat the beaker and mix the glycerin until it reaches a temperature of 150° C. This step ensures proper fluidity and facilitates the dissolution process.

Dissolve THCV: Once the glycol has reached the desired temperature, add THCV to the beaker. Allow the mixture to continue mixing until the THCV is fully dissolved. Add in cannabis and or hemp lipids and let dissolve.

Incorporate nano complex: Add the nano complex to the mixture in the beaker. Use a high shear mixer and continue mixing for 30 minutes. This ensures uniform distribution of the nano complex throughout the formulation, enhancing its effectiveness.

Integrate L-theanine: After the nano complex is well incorporated, add L-theanine to the mixture. Allow the solution to mix for an additional 10 minutes.

Add in water: Next, add the remaining water to the mixture. High-shear mix for 30 min

Turn off heat: Once all the components have been properly mixed and integrated, turn off the heat source.

Packaging: At this point, the formulation is complete and ready to be filled into the IV bag.

### Example 9

### 15 ml Oral Syringe Formulation

The following is an example method for making a formulation suitable for oral administration in a syringe.

Sanitize all lab equipment: Before starting the formulation process, ensure that all equipment, including beakers, mixers, and any other utensils, are thoroughly sanitized.

Prepare the mixture: Begin by adding glycol to a beaker. Heat the beaker and mix the glycol until it reaches a temperature of 150° C. This step ensures proper fluidity and facilitates the dissolution process.

Dissolve THCV: Once the glycol has reached the desired temperature, add THCV (tetrahydrocannabivarin) to the beaker. Allow the mixture to continue mixing until the THCV is fully dissolved. This step ensures proper dispersion of THCV in the solution.

Incorporate nano complex: Add the nano complex to the mixture in the beaker. Use a high shear mixer and continue mixing for 30 minutes. This ensures uniform distribution of the nano complex throughout the formulation, enhancing its effectiveness.

Integrate alpha GPC and L-theanine: After the nano complex is well incorporated, add alpha GPC and L-theanine to the mixture. Allow the solution to mix for an additional 10 minutes.

Mix in terpenes: Next, add the desired terpenes to the mixture. Mix the formulation to ensure even distribution of the terpenes throughout the solution.

Add in water: Next, add the remaining water to the mixture.

Turn off heat: Once all the components have been properly mixed and integrated, turn off the heat source.

Packaging: At this point, the formulation is complete and ready packaged into the Dial-a-dose syringe.

### Example 10

The following is an example method of administration for an IV solution.

Wash hands: Before handling any equipment or medication, make sure to wash your hands thoroughly with soap and water.

Gather supplies: Collect all the necessary supplies for IV administration, including the medication vial or ampule, a sterile syringe, an IV bag containing appropriate fluid, a spike or transfer device, alcohol swabs, and any additional equipment required.

Verify the medication: Check the medication label to ensure it is the correct medication, with the right dosage and expiration date. Confirm the compatibility of the medication with the fluid in the IV bag. If any doubts or discrepancies arise, consult a pharmacist or healthcare professional before proceeding. Prepare the IV bag: Suspend the IV bag containing the appropriate fluid on a suitable IV pole or hanger, ensuring it is securely positioned. If necessary, remove the protective cover from the injection port on the IV bag.

Prepare the medication: Follow the specific instructions provided with the medication. For a vial, clean the rubber stopper with an alcohol swab and use a sterile syringe to withdraw the prescribed amount of medication. For an ampule, carefully snap off the top and use a sterile syringe to draw up the required dose.

Spike the IV bag: Using a spike or transfer device, insert it into the injection port on the IV bag. Follow the manufacturer's instructions for proper insertion and secure attachment to prevent leakage.

Add the medication: With the IV bag properly spiked, inject the prepared medication from the syringe into the IV bag. Take care to avoid introducing air bubbles into the bag. Gently mix or swirl the IV bag to ensure proper mixing of the medication with the fluid.

Verify and label: Double-check that the medication has been correctly added to the IV bag. Label the IV bag with the medication name, dosage, and any other required information as per institutional or local protocols.

Prime the IV line: Open the IV line and prime it by allowing the fluid to flow.

### Example 11

The following is an example method for buccal administration.

Place the medication: With clean hands, gently place the medication or substance between the cheek and gums on the selected side of the mouth. Hold it in place with light pressure using your finger or by closing your mouth slowly. Avoid biting or chewing the medication.

Allow for absorption: Leave the medication or substance in place without swallowing or removing it for the recommended duration specified in the medication instructions or as advised by your healthcare provider. This allows time for absorption through the mucous membranes.

Avoid eating, drinking, or smoking: While the medication or substance is in place, avoid eating, drinking, or smoking, as this may interfere with the absorption process. Follow any specific instructions provided with the medication regarding food or drink restrictions.

Dispose of the residue: After the recommended duration, the medication or substance may have dissolved or lost its effectiveness. If any residue remains, it can be gently rinsed out with water or spit out, following appropriate disposal methods for the specific medication or substance.
Human: Take entire dose
Canine: under 10 lbs-(5 ml)/10 lbs-25 lbs-(10 ml)/25 lbs+ (15 ml) entire dose.

Do not exceed more than one syringe per day.

### Example 12

The following is an example method of administration for transdermal patch.

Clean the skin: Start by cleaning the area of skin where you will apply the transdermal patch or medication. Use mild soap and water, and make sure to dry the area thoroughly.

Select an appropriate location: Choose a location on the body where the skin is relatively thin, has good blood circulation, and is free from excessive hair, cuts, irritations, or rashes. Common areas include the upper arm, shoulder, back, abdomen, or thigh. Follow any specific instructions provided with the medication regarding the application site.

Apply the patch: If using a transdermal patch, remove it from its protective packaging. Peel off the backing and gently press the adhesive side onto the clean, dry skin. Apply firm pressure to ensure proper adhesion.

Rotate application sites: If you need to apply multiple transdermal patches, it's important to rotate the application sites to prevent irritation or skin damage. Avoid using the same spot repeatedly.

Avoid tampering: Once the transdermal patch is applied, avoid touching, rubbing, or tampering with it excessively. These actions can disrupt the delivery of the medication.

Remove and dispose of used patches properly: When it's time to remove a used transdermal patch, gently peel it off the skin. Fold the patch with the adhesive sides together and dispose of it properly according to the instructions provided with the medication. Some patches may need to be discarded in special containers to prevent accidental exposure.

Follow specific instructions: It's essential to carefully read and follow the specific instructions provided with the medication or transdermal patch you are using. Different medications and patches may have unique requirements, such as applying to specific body parts, avoiding water exposure, or other precautions.

Take the entire dose. Do not take more than one dose per day.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A formulation for the treatment of Cannabis toxicity comprising nano-emulsified THCV and an emulsifier.

2. The formulation of claim 1, further comprising L-theanine.

3. The formulation of claim 1, wherein the formulation is in the form of about a 2 oz beverage.

4. The formulation of claim 3, further comprising a supplement.

5. The formulation of claim 3, wherein the supplement is ashwagandha.

6. The formulation of claim 3, further comprising an acid.

7. The formulation of claim 6, wherein the acid is selected from the group consisting of citric acid, lemon juice, tartaric acid, white distilled vinegar, apple cider vinegar, ascorbic acid, and/or the like.

8. The formulation of claim 3 comprising 73.536%-99% water, 4.288%-6.432% monk fruit/erythritol, 0.12%-0.18% stevia, 0.104%-0.156% citric acid, 0.56%-0.84% lecithin, 0.056%-0.084 xanthan gum, 0.40%-0.6%, MCT/THCV, 0.072%-0.108% caffeine, 0.144%-0.216% L-theanine, 0.144%-0.216% choline, 0.008%-0.012% black pepper extract, 0.56%-0.84% natural and artificial flavors, 0.008%-0.012% emulsifying system.

9. The formulation of claim 1 in the form of a transdermal patch; the formulation comprising THCV, a nanoemulsifying system, alpha-GPC, L-theanine, one or more terpenes, and glycol.

10. The formulation of claim 1 in the form of liquid for IV administration; the formulation comprising THCV, a nanoemulsifying system, L-theanine, Cannabis-derived lipids, glycerin and water.

11. The formulation of claim 1 in the form of a liquid for administration with an oral syringe; the formulation comprising THCV, a nanoemulsifying system, alpha-GPC, L-theanine, one or more terpenes, glycerin, lipids, and water.

12. A method for treating Cannabis toxicity using the formulation of any of the preceding claims comprising: administering the formulation to a subject in an amount sufficient to alleviate one or more symptoms of Cannabis toxicity.

13. The method of claim 12, wherein the formulation is administered prior to consuming Cannabis.

14. The method of claim 12, wherein the formulation is administered after consuming Cannabis.

15. The method of claim 12, wherein the subject is a human.

16. The method of claim 12, wherein the subject is an animal.

17. The method of claim 12, wherein the administration step comprises delivering at least 5 mg of THCV.

18. A method for manufacturing the formulation of any of the preceding claims comprising THCV; comprising the steps of:
a. cleaning and sanitize a mixing vessel;
b. adding the water to the mixing vessel and begin homogenization operation;
c. adding one or more of the following ingredients to the mixing vessel: monkfruit/erythritol, stevia, citric acid, lecithin, xanthan gum, MCT/THCV, caffeine, L-theanine, choline, black pepper extract, natural and artificial flavors;
d. mixing with a high shear homogenizer until substantially homogeneous;
e. adding an emulsifying system as the final ingredient and mixing again with the homogenizer; and
f. distributing into container/package.
